(19) United States
(12) Patent Application Publication
(10) Pub. No.: US 2020/0237240 A1
(43) Pub. Date: Jul. 30, 2020

(54) VITAL-SIGN ESTIMATION APPARATUS AND CALIBRATION METHOD FOR VITAL-SIGN ESTIMATOR

(71) Applicant: MEDIATEK INC., Hsin-Chu (TW)

(72) Inventor: Chih-Ming FU, Hsinchu (TW)

(21) Appl. No.: 16/258,907

(22) Filed: Jan. 28, 2019

(51) Int. Cl. A61B 5/024; A61B 5/00; A61B 5/0205

(52) U.S. Cl. CPC A61B 5/02416; A61B 5/725; A61B 5/7221; A61B 5/7264; A61B 5/0205

(57) ABSTRACT

A vital-sign estimation apparatus is provided. The vital-sign estimation apparatus includes a physiological sensing device, a model generation circuit, and a vital-sign estimator. The physiological sensing device is configured to sense at least one physiological feature of an object to acquire at least one bio-signal. The model generation circuit provides a first reference model serving as an estimation mode. The vital-sign estimator generates vital-sign data according to the at least one bio-signal by using the estimation model. In response to the vital-sign estimation apparatus receiving calibration data, the model generation circuit changes the estimation model according to the calibration data thereby calibrating the vital-sign estimator.

### BACKGROUND OF THE INVENTION

### Field of the Invention

The invention relates to a vital-sign estimation apparatus, and more particularly to a calibration method for a vital-sign estimation apparatus.

### Description of the Related Art

With aging societies, more and more burden is placed on hospital resources. Moreover, cardiovascular diseases are increasing, as people age and stress increases for modern day living. For example, high blood pressure is a normal symptom of cardiovascular diseases. Thus, bio-signal self-measurement measurement devices have become an important target for development in the healthcare industry. Through sensing or detecting medically health information, such as electrocardiography (ECG), photoplethysmogram (PPG), heart rate, and blood pressure of patients in bio-signal self-measurement manners, the patients can monitor their own physiology status anytime, to relieve strain on hospital resources and provide needed medical attention to patients. As the bio-signal self-measurement measurement devices are used for a long time or when different patients use the same bio-signal self-measurement measurement device, the bio-signal self-measurement measurement devices needs to be calibrated. Generally, during a calibration operation of a bio-signal self-measurement measurement device, patient information data or vital-sign reference data is required and provided from an external device as reference for the calibration. However, the patient information data or vital-sign reference data may not be reliable, or the patient or medical staff or the operator of the bio-signal self-measurement measurement device cannot confirm that the patient information data or vital-sign reference data is reliable, which may result in that the bio-signal self-measurement measurement device cannot be calibrated correctly and accurately.

### BRIEF SUMMARY OF THE INVENTION

An exemplary embodiment of a vital-sign estimation apparatus is provided. The vital-sign estimation apparatus comprises a physiological sensing device, a model generation circuit, and a vital-sign estimator. The physiological sensing device is configured to sense at least one physiological feature of an object to acquire at least one bio-signal. The model generation circuit provides a first reference model serving as an estimation mode. The vital-sign estimator generates vital-sign data according to the at least one bio-signal by using the estimation model. In response to the vital-sign estimation apparatus receiving calibration data, the model generation circuit changes the estimation model according to the calibration data thereby calibrating the vital-sign estimator.

An exemplary embodiment of a calibration method for a vital-sign estimator is provided. The calibration method comprises steps of sensing at least one physiological feature of an object to acquire at least one bio-signal; providing a first reference model serving as an estimation mode; generating vital-sign data according to the at least one bio-signal by using the estimation model; receiving calibration data; and changing the estimation model according to the calibration data thereby calibrating the vital-sign estimator.

A detailed description is given in the following embodiments with reference to the accompanying drawings.

### BRIEF DESCRIPTION OF THE DRAWINGS

The invention can be more fully understood by reading the subsequent detailed description and examples with references made to the accompanying drawings, wherein:

FIG. 1 shows one exemplary embodiment of a vital-sign estimation apparatus;

FIG. 2 shows another exemplary embodiment of a vital-sign estimation apparatus;

FIG. 3 is a schematic diagram showing that a pulse wave transit time (PWTT) and a time period T_{R-R} according to an exemplary embodiment;

FIG. 4 shows a flow chart showing how to determine whether calibration data is reliable according to one exemplary embodiment;

FIG. 5 shows a flow chart showing how to determine whether calibration data is reliable according to another exemplary embodiment; and

FIG. 6 shows a flow chart showing how to determine whether calibration data is reliable according to another exemplary embodiment.

### DETAILED DESCRIPTION OF THE INVENTION

The following description is of the best-contemplated model of carrying out the invention. This description is made for the purpose of illustrating the general principles of the invention and should not be taken in a limiting sense. The scope of the invention is best determined by reference to the appended claims.

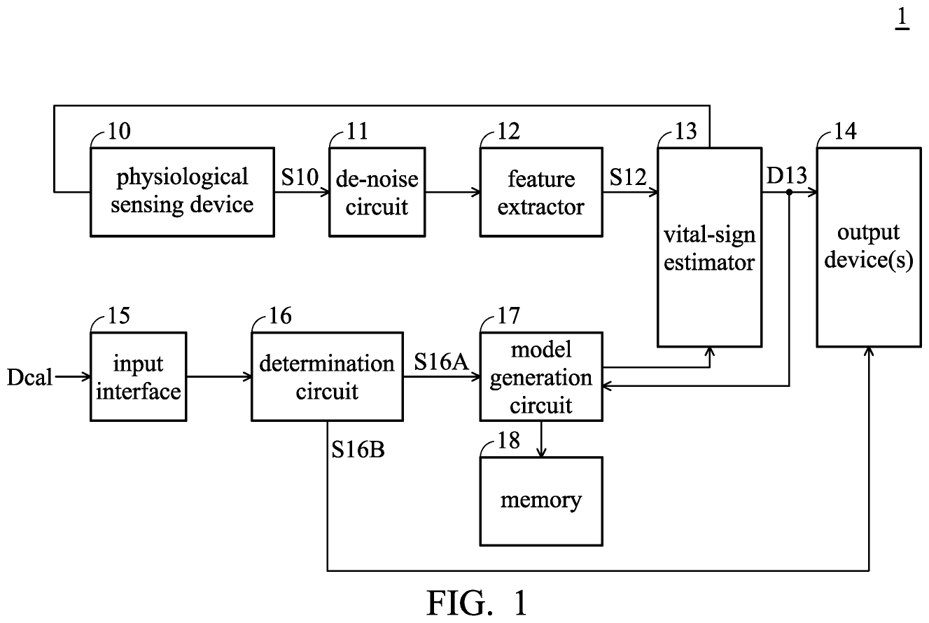

FIG. 1 shows one exemplary embodiment of a vital-sign estimation apparatus. As shown in FIG. 1, a vital-sign estimation apparatus 1 comprises a physiological sensing device 10, a de-noise circuit 11, a feature extractor 12, a vital-sign estimator 13, at least one output device 14, an input interface 15, a determination circuit 16, a model generation circuit 17, and a memory 18. The vital-sign estimation apparatus 1 can be a wearable device with a healthcare function, such as a smart watch, or a physiological monitor, such as an ExG monitor used to monitor at least one of electrocardiography (ECG), electroencephalograph (EEG), electromyography (EMG), electrooculography (EOG), electroretinogram (ERG), electrogastrography (EGG), and electroneurogram (ENG) of the object (such as the user), a photoplethysmogram (PPG) monitor, a heart rate monitor, or an oximeter. According to an embodiment, the physiological sensing device 10 may comprise electrodes and/or at least one light sensor to sense at least one physiological feature of the object who is wearing, holding or contacting the physiological sensing device 10. The at least one physiological feature may be at least one of the ECG, EEG, EMG, EOG, ERG, EGG, ENG, PPG, heart beats, or oxygen saturation of the object. The physiological sensing device 10 acquires at least one bio-signal S10 according to the sensed result. Based on the sensed physiological feature(s), the at least one bio-signal S10 comprises at least one of an ECG signal, an EGG signal, an EMG signal, an EOG signal, an ERG signal, an EGG signal, a PPG signal, or a heart-beat signal. In another embodiment, the physiological sensing device 10 may further comprise a motion sensor which is used to sense the motion or activity of the object. The motion sensor generates at least one bio-signal S10 according to the sensed motion or activity.

The bio-signal signals S10 are provided to the de-noise circuit 11. The de-noise circuit 11 performs a noise removal operation to remove noise of each bio-signal S10, and then the bio-signals S10 whose noise has removed are provided to the feature extractor 12. When the feature extractor 12 receives the bio-signals S10 through the de-noise circuit 11, the feature extractor 12 applies at least one feature extraction algorithm on the bio-signals S10 to generate corresponding feature signals S12. The feature extractor 12 transmits the feature signals S12 to the vital-sign estimator 13 for estimating vital-sign data D13 of the object according to at least one estimation model. The vital-sign data D13 may comprise, for example, at least one of a blood-pressure value, a heart-rate value, a sleep-phase indicator, an oxygen-saturation value, a heart-rate variability indicator, and an oxygen-saturation value. The estimated vital-sign data D13 is provided to the output device 14 to show at least one of a value, a diagram, and a waveform related to the estimated vital-sign data D13 or play a voice message about the estimated vital-sign data D13.

Referring to FIG. 1, the memory 18 stores a plurality of reference models. Some of the reference models are initially stored in the memory 18, and some of the reference models are previously built or generated by the model generation circuit 17 and then stored in the memory 18. According to an embodiment, the reference models are divided to several groups, and each group is related to the estimation of one type of vital signs. For example, the reference models are divided the groups which are related to, for example, the estimations of the blood-pressure value, the heart-rate value, the sleep-phase indicator, the oxygen-saturation value, the heart-rate variability indicator, and the oxygen-saturation value of the object respectively. For each group, there are several different reference models which are built or generated according to the physiological information, such as the age, gender, the height, the weight, the arm length, the drug-usage condition, and/or the disease information. For example, in each group, different ages are applied to build or generate different reference models.

The model generation circuit 17 accesses the memory 18 to read one reference model and provides the reference model to the vital-sign estimator 13 as the estimation model. In another embodiment, the estimation model used by the vital-sign estimator 13 is initially stored in a storage unit of the vital-sign estimator 13. During a calibration mode of the vital-sign estimation apparatus 1, calibration data Dcal is input to the vital-sign estimation apparatus 1 through the input interface 15. In an embodiment, the calibration data Dcal comprises vital-sign reference values, such as a blood-pressure reference value and a heart-rate reference value, and object information representing the characteristic and healthy condition of the object, such as the real age, gender, height, weight, arm length, drug-usage condition, and/or disease information of the object. The vital-sign reference values are obtained from at least one external device, such as an accurate sphygmomanometer, PPG monitor, and /or ECG monitor, during the calibration mode of the vital-sign estimation apparatus 1. Thus, the vital-sign reference values are also referred to as real vital-sign values, such as a real blood-pressure value and a real heart-rate value.

During the calibration operation, the physiological sensing device 10, the de-noise circuit 11, the feature extractor 12, the vital-sign estimator operates normally as in a measurement mode to generate the vital-sign data D13 of the vital-sign estimation apparatus 1. When the determination circuit 16 receives the calibration data Dcal through the input interface 15, the determination circuit 16 determines whether the calibration data Dcal is reliable and generates a calibration index S16A according to the determination result for the model generation circuit 17. When the determination circuit 16 determines that the calibration data Dcal is reliable, the model generation circuit 17 changes the estimation model which is currently used by the vital-sign estimator 13 according to the calibration index S16A. In an embodiment, the model generation circuit 17 modifies at least one parameter of the estimation model which is currently used by the vital-sign estimator 13. In another embodiment, the model generation circuit 17 provides another reference model which is stored in the memory 18 to the vital-sign estimator 13 as the estimation model (in other words, the estimation model which is used by the vital-sign estimator 13 is replaced with the reference model from the model generation circuit 17), thereby changing the estimation model. When the determination circuit 16 determines that the calibration data Dcal is not reliable, according to the calibration index S16A, the model generation circuit 17 decreases the weighting of a parameter of the estimation model related to the unreliable calibration data Dcal or provides another reference model whose parameters are not related to the unreliable calibration data Dcal to the vital-sign estimator 13 as the estimation model (in other words, the estimation model which is used by the vital-sign estimator 13 is replaced with the reference model whose parameters are not related to the unreliable calibration data Dcal). In an embodiment, the determination circuit 16 further generates a control signal S16B according to the determination result to the output device 14. The output device 14 may show a diagram or text message or play a voice message according to the control signal S16B to indicate whether the calibration data Dcal is reliable. In an embodiment, when determining that the calibration data Dcal is not reliable, the determination circuit 16 generates the control signal S 16B to control the output device 14 to show a warning message or play a warning sound.

The calibration index S16A is also generated according to the object information indicated by the calibration data Dcal. For example, the model generation circuit 17 determines what range the real age of the object is in and generates the calibration index S16A according to the determination result. If the estimation model which is used by the vital-sign estimator 113 is not appropriate to the determined range of the real age, the model generation circuit 17 selectively reads another reference model which is stored in the memory 18 according to the calibration index S16A and provides the reference model to the vital-sign estimator 13 as the estimation model (in other words, the estimation model which is used by the vital-sign estimator 13 is replaced with the reference model from the model generation circuit 17), thereby changing the estimation model. After the calibration mode, the vital-sign estimator 13 estimates the vital-sign data D13 of the object according to the changed estimation model during the subsequent measurement mode. If the estimation model which is used by the vital-sign estimator 113 is appropriate to the determined range of the real age, the mode generation circuit 17 does not change the estimation model.

According to the above embodiments, the vital-sign estimator 13 of the vital-sign estimation apparatus 1 can be calibrated by modifying or changing the estimation model according to the calibration data Dcal which is provided from the outside of the vital-sign estimation apparatus 1. Thus, as the vital-sign estimation apparatus 1 is used for a long time, the accuracy of the vital-sign estimation apparatus 1 is not decreased. Moreover, through the calibration operation, the vital-sign estimation apparatus 1 can estimate the vital signs of the objects with the different characteristics and healthy conditions by using appropriate estimation modes, thereby enhancing the accuracy of the vital-sign estimation apparatus 1.

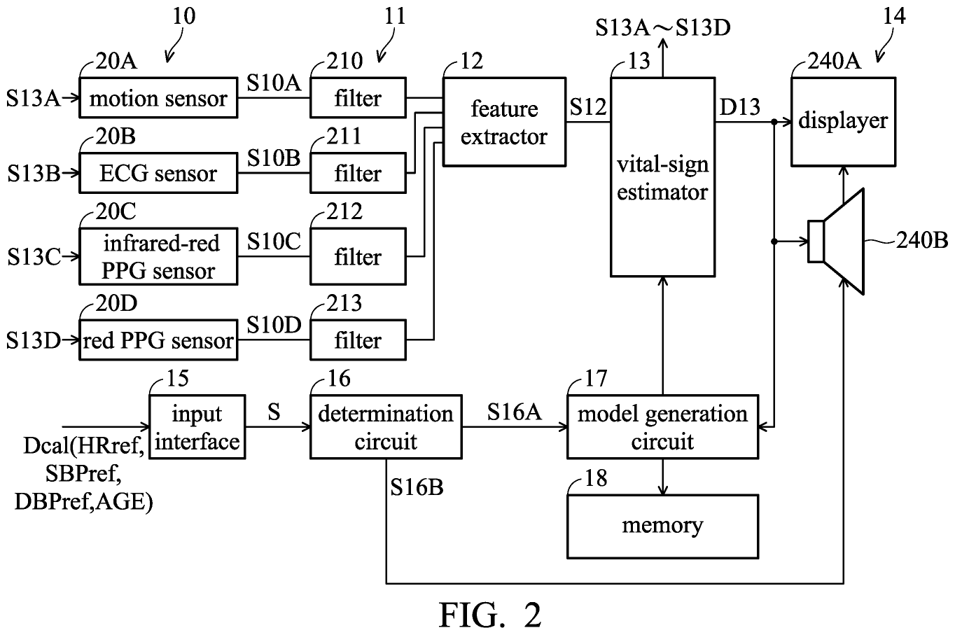

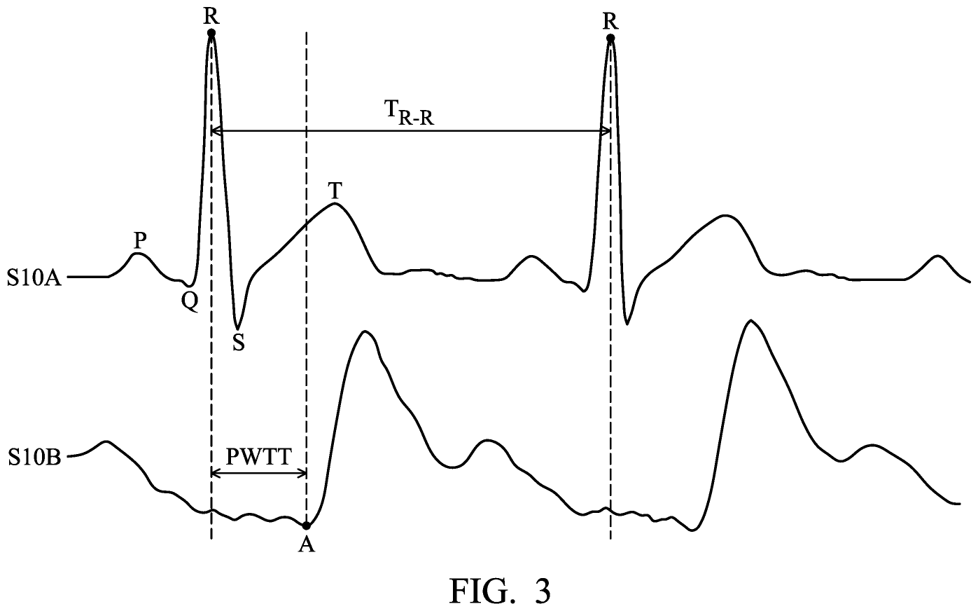

In the following paragraphs, an exemplary embodiment is provided for the detailed illustration. As shown in FIG. 2, the physiological sensing device 10 comprises a motion sensor 20A, an ECG sensor 20B, an infrared-red PPG sensor 20C, and a red PPG sensor 20C. The motion sensor 20A is configured to sense the motion or activity of the object and generate a bio-signal S10A according to the sensed result. In an embodiment, the motion sensor 20A comprises a G-sensor. The ECG sensor 20B senses the electrical activity of the heart (that is one of the above physiological features) of the object through electrodes contacting the skin of the object and generates a bio-signal S10B as shown in FIG. 3. The infrared-red PPG sensor 20C illuminates the skin of object (for example, the skin of the right wrist) by infrared-red light, measures changes in light absorption (that is one of the above physiological features), and generates a bio-signal S10C as shown in FIG. 3. Similarly, the red PPG sensor 20D illuminates the skin of the object (for example, the skin of the right wrist) by red light, measures changes in light absorption (that is one of the above physiological features), and generates a bio-signal S10D. The de-noise circuit 11 operates to noise of the bio-signals S10A-S10D. In an embodiment, the de-noise circuit 11 comprises filters 210-213 which are coupled to the motion sensor 20A, the ECG sensor 20B, the infrared-red PPG sensor 20C, and the red PPG sensor 20C to receive the bio-signals S10A-S10D, respectively. The filters 210-213 filter out the noise of the respective bio-signals S10A-S10D respectively through low-pass filtering operations, high-pass filtering operations, and/or band-pass filtering operations. The de-noise circuit 11 removing the noise of the bio-signals S10A-S10D through the filtering operations is given as an example. In other embodiments, the de-noise circuit 11 can perform other signal processing operations to filter out the noise of the bio-signals S10A-S10D. The, the bio-signals S10A-S10D whose noise has removed are provided to the feature extractor 12.

The feature extractor 12 applies at least one feature extraction algorithm on the bio-signals S10A-S10D to obtain corresponding feature signals S12. For example, the feature extractor 12 applies a feature extraction algorithm on the bio-signals S10B and S10C to calculate a pulse wave transit time (PWTT) between the bio-signals S10B and S10C. Referring to FIG. 3, the bio-signal S10B for ECG comprises a P wave, a Q wave, an R wave, an S wave, and a T wave for one cycle, and one cycle of the bio-signal S10B corresponds to one cycle of the bio-signal S10C for PPG. The feature extractor 12 detects the R wave occurs in one cycle of the bio-signal S10B and the lowest valley of the bio-signal S10C in the corresponding cycle and calculates the time period between the time point when the detected R wave of the bio-signal S10B occurs and the time point when the detected lowest valley of the bio-signal S10C. The calculated time period refers to the PWTT which indicates the time period when the pressure wave of the blood pressure is output to the right wrist from the heart. The PWTT is inversely proportional to the pulse transit speed and is also related to the blood pressure (one of the systolic blood pressure (SBP) and diastolic blood pressure (DBP)). In details, the higher pulse transit speed indicates the larger blood pressure, and the lower pulse transit speed indicates the less blood pressure. The feature extractor 12 generates one of the feature signals S12 according to PWTT. Moreover, the feature extractor 12 also detects the R waves occurring in two successive cycles of the bio-signal S10B and calculates the time period T_{R-R} between the time points when the two R waves of the bio-signal S10B occur. The time period T_{R-R} is related to the heart rate. The feature extractor 12 generates one of the feature signals S12 according to the time period T_{R-R}. The vital-sign estimator 13 receives the feature signals S12 and estimates the blood pressure (SBP or DBP) value and the heart-rate value of the object according to the PWTT and the time period T_{R-R} in indicated by the feature signals S12 by using the corresponding estimation models. In another embodiment, the feature extractor 12 further extracts the features of the bio-signal S10D, and the vital-sign estimator 13 estimates the blood pressure (SBP or DBP) value and the heart-rate value further according to the features of the bio-signal S10D. The estimated blood-pressure (SBP or DBP) value and the estimated heart-rate value serve as the vital-sign data D13, in other words, the vital-sign estimator 13 generates the vital-sign data D13 according to the estimated blood-pressure (SBP or DBP) value EBP and the estimated heart-rate value EHR. The vital-sign estimator 13 provides the vital-sign data D13 to the output device 14 and the model generation circuit 17. As shown in FIG. 2, the output device 14 comprises, for example, a displayer 240A and a speaker 240B. The displayer 240A is configured to show at least one of a value, a diagram, and a waveform related to the estimated vital-sign data D13. The speaker 240B is configured to play a voice message about the estimated vital-sign data D13. In the embodiment, the vital-sign extractor 17 also estimates the sleep-phase indicator to represent the sleep condition of the object according to the features of the bio-signal S10A. Moreover, in an embodiment, when determining that the calibration data Dcal is not reliable, the determination circuit 16 generates the control signal S16B to control the displayer 240A to show a diagram or text message, such as a warning message, and/or to control the speaker 240B to a voice message, such as a warning sound to indicate that the calibration data Dcal is not reliable.

According to an embodiment, the vital-sign extractor 13 may generate control signals S13A-S13B according to the estimated vital-sign data D13. For example, when the vital-sign extractor 13 determines that the estimated vital-sign data D13 is not accurate, it controls the sensors 20A-20D to adjust their operation conditions, such as the sensitivity, the light power, and so on.

In the embodiment, the model generation circuit 17 builds a reference model for blood-pressure estimation as BP=a/PWTT+b+c, wherein BP represents SBP or DBP which is estimated by the vital-sign estimator 13, PWTT is calculated by the feature extractor 12, the parameters “a” and “b” are constants, and the parameter “c” is an offset for calibration which is set initially or modified previously. The model generation circuit 17 provides the reference model for the blood-pressure estimation to the vital-sign estimator 13 as the estimation model. The vital-sign estimator 13 estimates the blood pressure (SBP or DBP) value according to the PWTT by using the estimation model BP=a/PWTT+b+c. In an embodiment, the offset is determined by the difference between a real blood-pressure value and an estimated blood-pressure value, such as the difference between a blood-pressure reference value which is obtained from an external device through the input interface 15 and a blood-pressure value which is estimated by the vital-sign estimator 13. In the cases for SPB estimation, the offset is determined by the difference between a SPB reference value which is obtained from an external device through the input interface 14 and a SPB value which is estimated by the vital-sign estimator 13. In order to calibrate the vital-sign estimator 13 during the current calibration mode, the model generation circuit 17 may modify the offset (the parameter “c”) of the reference model for blood-pressure estimation.

During the calibration mode, the calibration data Dcal is input to the vital-sign estimation apparatus 1 through the input interface 15. In the embodiment, the calibration data Dcal comprises a heart-rate reference value HRref, a SPB reference value SBPref, and DBP reference value DBPref which are measured and provided by at least one external device at the same time during the calibration mode. In order to modify the offset, the model generation circuit 17 calculates the difference between the SPB reference value SBPref and the estimated blood-pressure value EBP. However, if the SPB reference value SBPref is not reliable (for example, the SPB reference value SBPref is not accurate or wrong), the offset, which is calculated according to the SPB reference value SBPref, is not useful for the calibration of the vital-sign estimator 13. Thus, the determination circuit 16 has to determine whether the SPB reference value SBPref is reliable. When the determination circuit 16 determines that the calibration data Dcal is reliable, the model generation circuit 17 changes the estimation model which is currently used by the vital-sign estimator 13.

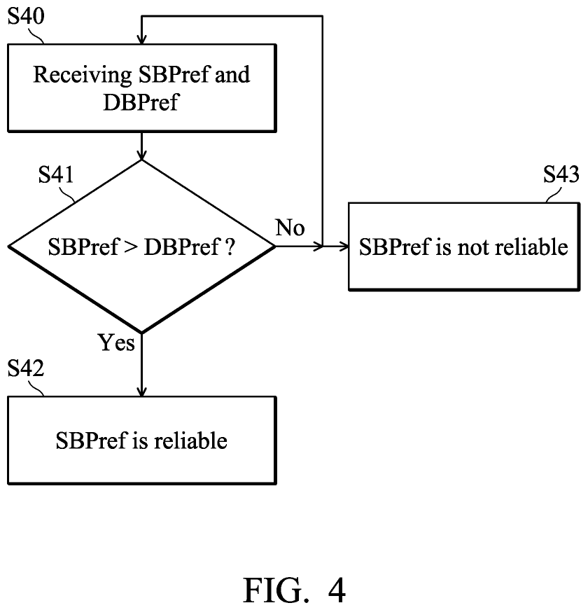

Referring to FIG. 4, a flow chart showing how to determine whether the SPB reference value SBPref is reliable is shown according to one exemplary embodiment. The determination circuit 16 receives the SPB reference value SBPref and the DBP reference value DBPref included in the calibration data Dcal (step S40) and determines whether the SPB reference value SBPref is larger than the DBP reference value DBPref (step S41). When the determination circuit 16 determines that the SPB reference value SBPref is larger than the DBP reference value DBPref (step S41-Yes), the determination circuit 16 determines that the SPB reference value SBPref is reliable (step S42). The model generation circuit 17 realizes that the SPB reference value SBPref is reliable according to the calibration index S16A and calculates the difference between the SPB reference value SBPref. Then, the model generation circuit 17 modifies the parameter “c” (the offset) to “c” according to the calculated difference thereby change the estimation model used by the vital-sign estimator 13. When the vital-sign estimation apparatus 1 enters the measurement mode next time, the vital-sign estimator 13 estimates the blood pressure by using the modified estimation model BP=a/PWTT+b+c'. When the determination circuit 16 determines that the SPB reference value SBPref and the RBP is not larger than the DBP reference value DBPref (step S41-No), the determination circuit 16 determines that the SPB reference value SBPref is not reliable (step S43), and the method proceeds to the step S40 to receive new calibration data. The model generation circuit 17 realizes that the SPB reference value SBPref is not reliable according to the calibration index S16A and then decreases the weighting of the parameter “c” or providing a reference model whose parameters are not related to the parameter “c” to the vital-sign estimator 13 as the estimation model.

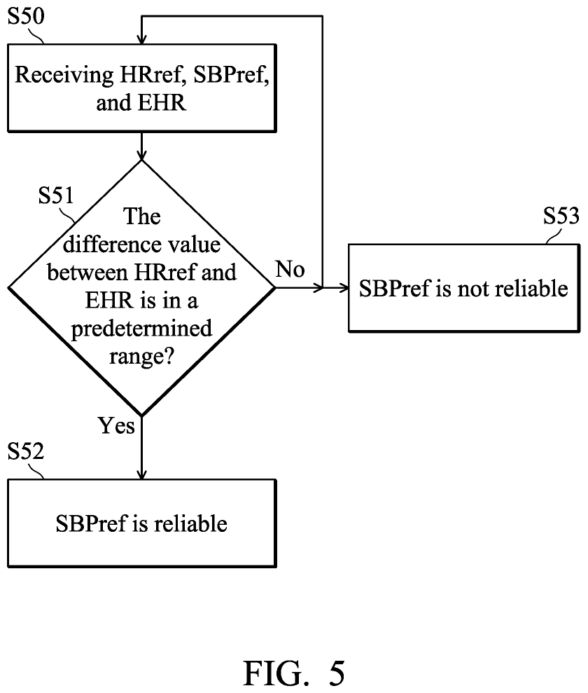

Referring to FIG. 5, a flow chart showing how to determine whether the SPB reference value SBPref is reliable is shown according to another exemplary embodiment. The determination circuit 16 receives the SPB reference value SBPref and the heart-rate reference value HRref included in the calibration data Dcal and further receives the estimated blood-pressure value EBP from the vital-sign estimator 13 (step S50) and determines whether the difference value of the of heart-rate reference value HRref and the estimated heart-rate value EHR is in a predetermined range (step S51). When the determination circuit 16 determines that the difference value of the of heart-rate reference value HRref and the estimated heart-rate value EHR is in the predetermined range (step S51-Yes), the determination circuit 16 determines that the SPB reference value SBPref is reliable (step S52). The model generation circuit 17 realizes that the SPB reference value SBPref is reliable according to the calibration index S16A and calculates the difference between the SPB reference value SBPref. Then, the model generation circuit 17 modifies the parameter “c” (the offset) to “c” according to the calculated difference thereby change the estimation model used by the vital-sign estimator 13. When the vital-sign estimation apparatus 1 enters the measurement mode next time, the vital-sign estimator 13 estimates the blood pressure by using the modified estimation model BP=a/PWTT+b+c'. When the determination circuit 16 determines that the difference value of the of heart-rate reference value HRref and the estimated heart-rate value EHR is not in the predetermined range (step S51-No), the determination circuit 16 determines that the SPB reference value SBPref is not reliable (step S53), and the method proceeds to the step S50 to receive new calibration data. The model generation circuit 17 realizes that the SPB reference value SBPref is not reliable according to the calibration index S 16A and then decreases the weighting of the parameter “c” or providing a reference model whose parameters are not related to the parameter “c” to the vital-sign estimator 13 as the estimation model.

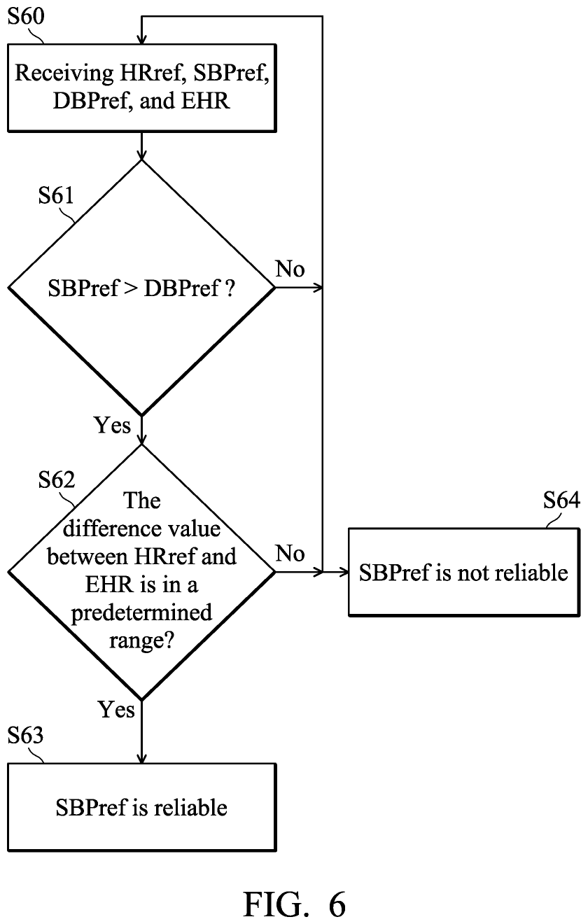

Referring to FIG. 6, a flow chart showing how to determine whether the SPB reference value SBPref is reliable is shown according to one exemplary embodiment. The determination circuit 16 receives the SPB reference value SBPref, the DBP reference value DBPref, and the heart-rate reference value HRref included in the calibration data Dcal and further receives the estimated blood-pressure value EBP from the vital-sign estimator 13 (step S60) and determines whether the SPB reference value SBPref is larger than the DBP reference value DBPref (step S51). When the determination circuit 16 determines that the SPB reference value SBPref is larger than the DBP reference value DBPref (step S61-Yes), the determination circuit 16 determines whether the difference value of the of heart-rate reference value HRref and the estimated heart-rate value EHR is in a predetermined range (step S62). When the determination circuit 16 determines that the SPB reference value SBPref and the RBP is not larger than the DBP reference value DBPref (step S61-No), the method proceeds to the step S60 to receive new calibration data. In the step S62, when the determination circuit 16 determines that the difference value of the of heart-rate reference value HRref and the estimated heart-rate value EHR is in the predetermined range (step S62-Yes), the determination circuit 16 determines that the SPB reference value SBPref is reliable (step S63). The model generation circuit 17 realizes that the SPB reference value SBPref is reliable according to the calibration index S16A and calculates the difference between the SPB reference value SBPref. Then, the model generation circuit 17 modifies the parameter “c” (the offset) to “c” according to the calculated difference thereby change the estimation model used by the vital-sign estimator 13. When the vital-sign estimation apparatus 1 enters the measurement mode next time, the vital-sign estimator 13 estimates the blood pressure by using the modified estimation model BP=a/PWTT+b+c'. When the determination circuit 16 determines that the difference value of the of heart-rate reference value HRref and the estimated heart-rate value EHR is not in the predetermined range (step S62-No), the determination circuit 16 determines that the SPB reference value SBPref is not reliable (step S63), and the method proceeds to the step S50 to receive new calibration data. The model generation circuit 17 realizes that the SPB reference value SBPref is not reliable according to the calibration index S 16A and then decreases the weighting of the parameter “c” or providing a reference model whose parameters are not related to the parameter “c” to the vital-sign estimator 13 as the estimation model.

While the invention has been described by way of example and in terms of the preferred embodiments, it is to be understood that the invention is not limited to the disclosed embodiments. On the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A vital-sign estimation apparatus comprising:
a physiological sensing device configured to sense at least one physiological feature of an object to acquire at least one bio-signal;
a model generation circuit providing a first reference model serving as an estimation model; and
a vital-sign estimator generating vital-sign data according to the at least one bio-signal by using the estimation model;
wherein in response to the vital-sign estimation apparatus receiving calibration data, the model generation circuit changes the estimation model according to the calibration data thereby calibrating the vital-sign estimator.

2. The vital-sign estimation apparatus as claimed in claim 1, further comprising:
a feature extractor receiving the at least one bio-signals and extracting features of the at least one bio-signals,
wherein the vital-sign estimator generates the vital-sign data according to the features of the at least one bio-signals by using the estimation model.

3. The vital-sign estimation apparatus as claimed in claim 1, further comprising:
a determination circuit receiving the calibration data, determining whether the calibration data is reliable to generate a first determination result, and generating a calibration index according to the first determination result for the model generation circuit,
wherein in response to the determination circuit determining that the calibration data is reliable, the model generation circuit modifies at least one parameter of the first reference model according to the calibration index thereby changing the estimation model.

4. The vital-sign estimation apparatus as claimed in claim 3,
wherein the vital-sign data comprises an estimated heart-rate value and an estimated blood-pressure value, and the calibration data comprises a heart-rate reference value and a blood-pressure reference value; and
wherein the determination circuit determines whether a difference value between the estimated heart-rate value and the heart-rate reference value is in a predetermined range to generate a second determination result and determines whether the blood-pressure reference value is reliable according to the second determination result.

5. The vital-sign estimation apparatus as claimed in claim 4, wherein in response to the determination circuit determining that the difference value between the estimated heart-rate value and the heart-rate reference value is in the predetermined range, the determination circuit determines that the blood-pressure reference value is reliable, and the model generation circuit modifies the at least one parameter of the first reference model by the blood-pressure reference value according to the calibration index.

6. The vital-sign estimation apparatus as claimed in claim 3, wherein in response to the determination circuit determining that the calibration data is not reliable, the model generation circuit, according to the calibration index, decreases weighting of a parameter of the first reference model related to the calibration data or providing a second reference model whose parameters are not related to the calibration data to the vital-sign estimator as the estimation model instead of the first reference model.

7. The vital-sign estimation apparatus as claimed in claim 3, further comprising:
an output device coupled to the determination circuit,
wherein in response to the determination circuit determining that the calibration data is not reliable, the output device shows a diagram or text message or play a voice message to indicate that the calibration data is not reliable.

8. The vital-sign estimation apparatus as claimed in claim 1, further comprising:
a determination circuit receiving the calibration data, determining object information indicated by the calibration data to generate a first determination result, and generating a calibration index according to the first determination result for the model generation circuit,
a memory storing a plurality of reference models,
wherein the model generation circuit selectively reads one of the plurality of reference models from the memory as a second reference model acceding to the calibration index, and the second reference model replaces the first reference model as the estimation model thereby changing the estimation model.

9. The vital-sign estimation apparatus as claimed in claim 8, wherein the object information indicated by the calibration data comprises at least one of age, gender, height, weight, arm length, a drug-usage condition, and disease information.

10. The vital-sign estimation apparatus as claimed in claim 1, wherein the at least one bio-signal comprises at least one of an electrocardiography (ECG) signal, an electroencephalograph (EEG) signal, an electromyography (EMG) signal, an electrooculography (EOG) signal, an electroretinogram (ERG) signal, an electrogastrography (EGG) signal, an electroneurogram (ENG), a photoplethysmogram (PPG) signal, and a heart-beat signal.

11. The vital-sign estimation apparatus as claimed in claim 1, wherein the vital-sign data comprises at least one of a blood-pressure value, a heart-rate value, a sleep-phase indicator, an oxygen-saturation value, and heart-rate variability indicator.

12. A calibration method for a vital-sign estimator 13 comprising:
sensing at least one physiological feature of an object to acquire at least one bio-signal;
providing a first reference model serving as an estimation model;
generating vital-sign data according to the at least one bio-signal by using the estimation model;
receiving calibration data; and
changing the estimation model according to the calibration data thereby calibrating the vital-sign estimator.

13. The calibration method for as claimed in claim 12, wherein generating the vital-sign data according to the at least one bio-signal by using the estimation model comprises:
extracting features of the at least one bio-signals; and
generating the vital-sign data according to the features of the at least one bio-signals by using the estimation model.

14. The calibration method as claimed in claim 12, wherein changing the estimation model according the calibration data comprises:
determining whether the calibration data is reliable to generate a first determination result;
generating a calibration index according to the first determination result,
in response to determining that the calibration data is reliable, modifying at least one parameter of the first reference model according to the calibration index thereby changing the estimation model.

15. The calibration method as claimed in claim 14,
wherein the vital-sign data comprises an estimated heart-rate value and an estimated blood-pressure value, and the calibration data comprises a heart-rate reference value and a blood-pressure reference value; and
wherein determining whether the calibration data is reliable comprises:
determining whether a difference value between the estimated heart-rate value and the heart-rate reference value is in a predetermined range to generate a second determination result; and
determining whether the blood-pressure reference value is reliable according to the second determination result.

16. The calibration method as claimed in claim 15,
wherein determining whether the calibration data is reliable comprises:
in response to determining that the difference value between the estimated heart-rate value and the heart-rate reference value is in the predetermined range, determining that the blood-pressure reference value is reliable, and
wherein in response to determining that the calibration data is reliable, the at least one parameter of the first reference model is modified by the blood-pressure reference value according to the calibration index.

17. The calibration method as claimed in claim 14, wherein changing the estimation model according the calibration data comprises:
in response to determining that the calibration data is not reliable, decreasing weighting of a parameter of the first reference model related to the calibration data or providing a second reference model whose parameters are not related to the calibration data to the vital-sign estimator instead of the first reference model according to the calibration index.

18. The calibration method as claimed in claim 14, further comprising:
in response to determining that the calibration data is not reliable, showing a diagram or text message or playing a voice message to indicate that the calibration data is not reliable.

19. The calibration method as claimed in claim 12, further comprising:
storing a plurality of reference models in a memory,
wherein changing the estimation model according the calibration data comprises:
determining object information indicated by the calibration data to generate a first determination result;
generating a calibration index according to the first determination result;
selectively reading one of the plurality of reference models from the memory as a second reference model acceding to the calibration index; and
providing the second reference model to replace the first reference model as the estimation model thereby changing the estimation model.

20. The calibration method as claimed in claim 19, wherein the object information indicated by the calibration data comprises at least one of age, gender, height, weight, arm length, a drug-usage condition, and disease information.

21. The calibration method as claimed in claim 12, wherein the at least one bio-signal comprises at least one of an electrocardiography (ECG) signal, an electroencephalograph (EEG) signal, an electromyography (EMG) signal, an electrooculography (EOG) signal, an electroretinogram (ERG) signal, an electrogastrography (EGG) signal, an electroneurogram (ENG), a photoplethysmogram (PPG) signal, and a heart-beat signal.

22. The calibration method as claimed in claim 12, wherein the vital-sign data comprises at least one of a blood-pressure value, a heart-rate value, a sleep-phase indicator, an oxygen-saturation value, and heart-rate variability indicator.
